SEQUENCE LISTING (8 sequences)

SEQ ID NO: 1 - protein, 7 aa, Artificial Sequence
  Standard peptide sequence to test antibody reactivity
QETCIQG

SEQ ID NO: 2 - protein, 8 aa, Artificial Sequence
  Elongated peptide sequence
QETCIQGA

SEQ ID NO: 3 - protein, 6 aa, Artificial Sequence
  Truncated peptide sequence
QETCIQ

SEQ ID NO: 4 - protein, 10 aa, Artificial Sequence
  Non-sense peptide sequence
GLRPGSEYTV

SEQ ID NO: 5 - protein, 11 aa, Artificial Sequence
  Non-sense coater sequence
  SITE 11: Biotinylated
GLRPGSEYTVK

SEQ ID NO: 6 - protein, 10 aa, Artificial Sequence
  Elongated peptide sequence
KECQETCIQG

SEQ ID NO: 7 - protein, 10 aa, Artificial Sequence
  Immunogenic peptide sequence
  SITE 1: Keyhole Limpet Hemocyanin
CGGQETCIQG

SEQ ID NO: 8 - protein, 7 aa, Artificial Sequence
  Screening peptide sequence
  SITE 1: Biotinylated
QETCIQG

(19) United States
(12) Patent Application Publication
(10) Pub. No.: US 2023/0358752 A1
(43) Pub. Date: Nov. 9, 2023

(54) Assay for Assessing Cancer

(71) Applicant: Nordic Bioscience A/S, Herlev (DK)

(72) Inventors: Federica Genovese, København (DK); Nicholas Willumsen, Gentofte (DK); Alexander Lynge Reese-Petersen, Lynge (DK); Shu Sun, Rødovre (DK); Morten Asser Karsdal, København Ø (DK)

(73) Assignee: Nordic Bioscience A/S, Herlev (DK)

(21) Appl. No.: 18/025,704

(22) PCT Filed: Sep. 10, 2021
(86) PCT No.: PCT/EP2021/074996

§ 371 Date: Mar. 10, 2023

(30) Foreign Application Priority Data
Sep. 11, 2020 (GB) 2014323.6

(51) Int. Cl. G01N 33/574

(52) U.S. Cl. CPC G01N 33/57484; G01N 2333/78

(57) ABSTRACT

Described herein are immunoassay methods for detecting and/or monitoring a cancer in a patient by contacting a biofluid sample from a patient with a monoclonal antibody that specifically binds to a C-terminal epitope of type XXVIII collagen, and detecting and determining the amount of binding between the monoclonal antibody and peptides in the sample.

### FIELD OF THE INVENTION

The present invention relates to immunoassays for detecting and/or monitoring a cancer in a patient. The cancer may in particular be lung, breast, colorectal or pancreatic cancer. The immunoassay may in certain embodiments be for detecting the stage of a cancer in a patient or determining the prognosis of a cancer in a patient.

### BACKGROUND

Type XXVIII collagen is poorly described in literature but research entailing its physical role is slowly emerging. It is mainly located in peripheral nerves and dorsal root ganglia, but is also found in the skin (1,2). Type XXVIII collagen is a beaded collagen that structurally resembles type VI collagen, with two von Willebrand factor A domains flanking a 528 amino acid collagenous domain (3). Type XXVIII collagen was found in very low levels in healthy lung tissue but was overexpressed in bleomycin-induced lung injury (4), which could indicate that cells expressing type XXVIII collagen might be involved in tissue repair processes. Type XXVIII collagen has also previously been seen to be upregulated in mouse hepatocarcinoma (5).

### SUMMARY

The present inventors have now determined that collagen type XXVIII formation is upregulated in cancers, such as in particular lung, breast, colorectal or pancreatic cancers, and have developed a competitive ELISA utilizing monoclonal antibodies targeting the C-terminal end of type XXVIII collagen that can be used to detect and/or monitor a cancer and determine the stage of and/or prognosis of a cancer.

Accordingly, the present invention provides a method of immunoassay for detecting and/or monitoring a cancer in a patient, the method comprising:
(i) contacting a biofluid sample from a patient with a monoclonal antibody that specifically binds to a C-terminal epitope of type XXVIII collagen,
(ii) detecting and determining the amount of binding between said monoclonal antibody and peptides in the sample or samples, and
(iii) correlating said amount of binding of said monoclonal antibody as determined in step (ii) with values associated with normal healthy subjects and/or values associated with known cancer severity and/or values obtained from said patient at a previous time point and/or a predetermined cut-off value.

The immunoassay may be, but is not limited to, a competition assay or a sandwich assay. The immunoassay may, for example, be a radioimmunoassay or an enzyme-linked immunosorbent assay (ELISA). Such assays are techniques known to the person skilled in the art.

The cancer may in certain embodiments be lung, breast, colorectal, ovarian and/or pancreatic cancer. In particular, the cancer may be lung, breast, colorectal and/or pancreatic cancer.

The method may in certain embodiments be a method for detecting the severity of a cancer in a patient. For example, the method may be a method for detecting the stage of a cancer in a patient, and/or a method of determining the prognosis of a cancer in a patient (such as for example determining the likely survival time or probability of survival of the patient).

In certain embodiments, the patient may for example be a patient undergoing a therapy for the cancer, and the method may comprise monitoring the cancer in the patient.

The patient biofluid sample may be, but is not limited to, blood, serum, plasma, urine or a supernatant from cell or tissue cultures. Preferably the biofluid is serum or plasma, most preferably serum.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the methods of the present invention, a monoclonal antibody comprising any constant region known in the art can be used. Human constant light chains are classified as kappa and lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgG1, IgG2, IgG3, and IgG4. The monoclonal antibody may preferably be of the IgG isotype, including any one of IgG1, IgG2, IgG3 or IgG4.

The CDR of an antibody can be determined using methods known in the art such as that described by Kabat et al. Antibodies can be generated from B cell clones as described in the examples. The isotype of the antibody can be determined by ELISA specific for human IgM, IgG or IgA isotype, or human IgG1, IgG2, IgG3 or IgG4 subclasses. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

In some embodiments of the methods according to the invention, the biofluid sample is contacted with a monoclonal antibody which specifically binds to a C-terminus amino acid sequence QETCIQG (SEQ ID NO: 1) (also referred to herein as “PRO-C28”). Preferably said monoclonal antibody does not recognize or specifically bind to an elongated version of said C-terminus amino acid sequence which is QETCIQGA (SEQ ID NO: 2). Preferably said monoclonal antibody does not recognize or specifically bind to a truncated version of said C-terminus amino acid sequence which is QETCIQ (SEQ ID NO: 3).

Preferably, the ratio of the affinity of said antibody for the C-terminus amino acid sequence QETCIQG (SEQ ID NO: 1) to the affinity of said antibody for the elongated C-terminus amino acid sequence QETCIQGA (SEQ ID NO: 2) is at least 10 to 1, and more preferably is at least 50 to 1, at least 100 to 1, at least 500 to 1, at least 1,000 to 1, at least 10,000 to 1, at least 100,000 to 1, or at least 1,000,000 to 1.

Preferably, the ratio of the affinity of said antibody for the C-terminus amino acid sequence QETCIQG (SEQ ID NO: 1) to the affinity of said antibody for the truncated C-terminus amino acid sequence QETCIQ (SEQ ID NO: 3) is at least 10 to 1, and more preferably is at least 50 to 1, at least 100 to 1, at least 500 to 1, at least 1,000 to 1, at least 10,000 to 1, at least 100,000 to 1, or at least 1,000,000 to 1.

As used herein the term “C-terminus” refers to a C-terminal peptide sequence at the extremity of a polypeptide, i.e. at the C-terminal end of the polypeptide, and is not to be construed as meaning in the general direction thereof.

Monoclonal antibodies that specifically bind to the C-terminus amino acid sequence QETCIQG (SEQ ID NO: 1) can be generated via any suitable techniques known in the art. For example, the monoclonal antibody may be raised against a synthetic peptide having the amino acid sequence QETCIQG (SEQ ID NO: 1), such as for example by: immunizing a rodent (or other suitable mammal) with a synthetic peptide consisting of the sequence QETCIQG (SEQ ID NO: 1), which optionally may linked to an immunogenic carrier protein (such as keyhole limpet hemocyanin), isolating and cloning a single antibody producing cell, and assaying the resulting monoclonal antibodies to ensure that they have the desired specificity. An exemplary protocol for producing a monoclonal antibody that that specifically bind to the C-terminus amino acid sequence QETCIQG (SEQ ID NO: 1) is described infra.

In some embodiments of the methods according to the invention, the amount of binding of the monoclonal antibody specific for the C-terminal epitope of type XXVIII collagen is correlated with values associated with normal healthy subjects and/or with values associated with known cancer severity and/or with values obtained from the patient at a previous point in time.

As used herein the term “values associated with normal healthy subjects and/or values associated with known cancer severity” means standardised quantities determined by the method described supra for subjects considered to be healthy, i.e. without cancer, and/or standardised quantities determined by the method described supra for subjects known to have a cancer with a known severity. Thus, for example, where the method is a method for detecting lung, breast or colorectal cancer, the amount of binding of the monoclonal antibody may be correlated with standardised quantities determined by the method for healthy subjects and/or with standardised quantities determined by the method for subjects known to have said cancer with a known severity.

In some embodiments of the method according to the invention, the amount of binding of the monoclonal antibody specific for the C-terminal epitope of type XXVIII collagen is correlated with one or more predetermined cut-off values.

As used herein the “cut-off value” means an amount of binding that is determined statistically to be indicative of a high likelihood of a cancer, or of high likelihood a cancer of a particular stage or other level of severity, in a patient. For example, a cut-off value may be chosen such that a measured value of biomarker binding in a patient sample that is at or above the statistical cut-off value corresponds to at least a 70% probability, preferably at least an 80% probability, preferably at least an 85% probability, more preferably at least a 90% probability, and most preferably at least a 95% probability of the presence or likelihood of a cancer or of a particular stage or other level of severity of the cancer in the patient.

The predetermined cut-off value for the amount of binding of the monoclonal antibody specific for the C-terminal epitope of type XXVIII collagen may for example be at least 50 ng/mL, and more preferably may be at least 60 ng/mL, at least 70 ng/mL, at least 80 ng/mL, at least 90 ng/mL or at least 100 ng/mL. In this regard, through the use of statistical analyses it has been found that a measured amount of binding of the monoclonal antibody specific for the C-terminal epitope of type XXVIII collagen at or above said cut-off values (e.g. a measured amount of 60 ng/mL or greater where a cut-off value of at least 60 ng/mL is for example used) may be indicative of various cancers. It has also been found that higher measured amounts of binding of the monoclonal antibody correlate statistically with later stages of cancer and worse prognoses. Accordingly, where the method is a method for detecting the stage of a cancer in a patient, cut-off values for each stage of cancer may be used, with higher cut-off values being used for later stages. Likewise, where the method is a method for determining the prognosis of a cancer in a patient, cut-off values may be used that correspond with certain prognoses (such as likely periods of survival or chances of survival). By using such statistical cut-off values it is possible to utilise the method of the invention to give a diagnosis with a high level of confidence. Applying such statistical cut-off values are particularly advantageous as it results in a standalone diagnostic assay; i.e. it removes the need for any direct comparisons with healthy individuals and/or patients with known cancer severity in order to arrive at a diagnostic conclusion. This may also be particularly advantageous when utilising the assay to evaluate patients that already have medical signs or symptoms that are generally indicative of cancer (e.g. as determined by a physical examination and/or consultation with a medical professional) as it may act as a quick and definitive tool for corroborating the initial diagnosis and thus potentially remove the need for more invasive procedures, and expedite the commencement of a suitable treatment regimen. It may also avoid the need for a lengthy hospital stay. In the particular case of cancer, an expedited diagnosis may result in the disease being detected at an earlier stage, which may in turn improve overall chances of survival.

### FIGURES

FIG. 1: Antibody specificity. Reactivity was tested using the standard peptide (QETCIQG (SEQ ID NO: 1)), an elongated peptide (QETCIQGA (SEQ ID NO: 2)), a non-sense peptide (GLRPGSEYTV (SEQ ID NO: 4)) and a non-sense coater (GLRPGSEYTV-K-Biotin (SEQ ID NO: 5)).

FIG. 2: Levels of PRO-C28 in serum of healthy controls and patients with cancer. Levels of PRO-C28 were significantly elevated in mixed samples from cancer patients (Asterand, P=0.002) and in lung cancer samples (Proteogenex, P<0.0001) (Figure A). Statistical difference was assessed using ANOVA with Dunnett's multiple comparisons test. PRO-C28 was able to significantly discriminate between healthy individuals and a cohort of mixed (Figure B) and lung cancer (Figure C) patients (P=0.0007, P<0.0001, respectively).

FIG. 3: Levels of PRO-C28 in serum from healthy controls and patients with various solid tumors defined by organ.

FIG. 4: Levels of PRO-C28 in serum from various cancer patients grouped by cancer stage. The line is fitted from linear regression analysis.

FIG. 5: Kaplan Meier overall survival curves for pancreatic cancer patients undergoing chemotherapy, the patients being divided and grouped into tertiles (Q1, Q2 and Q3) according to pre-treatment levels of PRO-C28.

### EXAMPLES

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

In the following examples, the following materials and methods were employed.

### Materials and Methods

All reagents used for the experiments were high quality standards from companies such as Sigma Aldrich (St. Louis, MO, USA) and Merck (Whitehouse Station, NJ, USA). The synthetic peptides used for immunization and assay development were purchased from the Genscript (New Jersey, USA).

### Generation of Monoclonal Antibodies Targeting PRO-C28

Monoclonal antibodies targeting the C-terminal end of collagen type XXVIII were generated by raising antibodies against the 7 amino acid sequence QETCIQG (SEQ ID NO: 1) (“PRO-C28”) found at the C-terminus of collagen type XXVIII. This 7 amino acid sequence was chosen rather than a longer sequence, such as the 10 amino acid C-terminus sequence KECQETCIQG (SEQ ID NO: 6), in order to reduce the number of cystein residues and so avoid the formation of a Cys-cys bridge in the immunogenic peptide that was being used to generate the antibodies.

The protocol used for generation of monoclonal antibodies targeting PRO-C28 was as follows.

Immunization of 6-7 week old female Balb/C mice (weighing 14-18 g) was initiated by subcutaneous injection of 200 μL emulsified antigen solution comprising 100 μg immunogenic peptide (KLH-CGG-QETCIQG (SEQ ID NO: 7), where ‘KHL’ indicates keyhole limpet hemocyanin and CGG is a conjugation linker) in Stimune Immunogenic Adjuvant (SPECOL)(Cat #7925000, Invitrogen). The immunizations were repeated every 2^{nd} week until stable serum antibody titer levels were reached. The mouse with the highest serum titer and best inhibition was selected for fusion and rested for at least three weeks following the last immunization. Subsequently, the mouse was boosted intravenously with 100 μg immunogenic peptide in 100 μL 0.9% NaCl solution three days before isolation of the spleen for cell fusion. To produce hybridoma cells, the mouse spleen cells were fused with SP2/0 myeloma cells as described by Gefter et al. The hybridoma cells were cloned in culture dishes using the semi-solid medium method. The clones were then plated into 96-well microtiter plates for further growth, and the limiting dilution method was applied to promote monoclonal growth. Indirect ELISA performed on streptavidin-coated plates was used for the screening of supernatant reactivity. Biotin-QETCIQG (SEQ ID NO: 8) was used as the screening peptide, while the standard peptide (QETCIQG (SEQ ID NO: 1)), an elongated peptide (QETCIQGA (SEQ ID NO: 2)), a non-sense peptide (GLRPGSEYTV (SEQ ID NO: 4)) and a non-sense coater (GLRPGSEYTV-K-Biotin (SEQ ID NO: 5)) were used for further testing of the specificity of the clones. Supernatant was collected from the hybridoma cells, and purified using HiTrap affinity columns (GE Healthcare Life Science, Little Chalfront, Buckinghamshire, UK) according to manufacturer's instructions. All animals were treated according to the guidelines for animal welfare.

### Clone Selection and Characterization

The best antibody producing hybridomas were screened for reactivity towards the standard peptide (QETCIQG (SEQ ID NO: 1)) in the competitive ELISA described below, and the clone showing the most reactivity was selected for production of monoclonal antibodies targeting PRO-C28. Antibody specificity was tested using the standard peptide (QETCIQG (SEQ ID NO: 1)), elongated peptide (QETCIQGA (SEQ ID NO: 2)), non-sense peptide (GLRPGSEYTV (SEQ ID NO: 4)) and non-sense coater (GLRPGSEYTV-K-Biotin (SEQ ID NO: 5)). The isotype of the monoclonal antibody was determined using the Clonotyping System-HRP kit, cat. 5300-05 (Southern Biotech, Birmingham, AL, USA).

### PRO-C28 ELISA

A 96-well streptavidin-coated ELISA plate from Roche, cat.11940279, was coated with 100 μL/well of the biotinylated peptide Biotin-QETCIQG (SEQ ID NO: 8) dissolved in assay buffer (25 mM TBS-BTE+2 g/l NaCl, pH 8), incubated for 30 min at 20° C. in the dark with shaking, and subsequently washed 5 times in washing buffer (20 mM Tris, 50 mM NaCl, pH 7.2). Thereafter 20 μl of peptide calibrator or sample were added to appropriate wells, followed by 100 μl of purified antibody solution (monoclonal antibodies specific for PRO-C28 dissolved in assay buffer), and incubated for 1 hour at 20° C. with shaking, followed by washing 5 times in washing buffer. Next, 100μL secondary antibody solution (horseradish peroxidase (HRP) labeled anti-mouse antibodies dissolved in the same assay buffer as used for the monoclonal antibody specific for PRO-C28) was added to each well, incubated for 1 hour at 20° C. with shaking, followed by washing 5 times in washing buffer. Finally, 100 μl tetramethylbenzinidine (TMB) (Kem-En-Tec cat.: 438OH) was added to each well, the plate was incubated for 15 min at 20° C. in the dark, and in order to stop the reaction 100 μl of stopping solution (1% H₂SO₄) was added and the plate was then analyzed in the ELISA reader at 450 nm with 650 nm as the reference (Molecular Devices, SpectraMax M, CA, USA). A calibration curve was plotted using a 4-parametric mathematical fit model.

### Technical Evaluation of PRO-C28 ELISA

A twofold dilution of human serum, human urine and EDTA, heparin or citrate treated human plasma samples (four of each type of sample) was used to assess the linearity. The linearity was calculated as a percentage of recovery of the undiluted sample.

The intra- and inter-assay variation was determined by 10 independent runs of five quality control (QC) and two kit controls run in double determinations.

Accuracy of the assay was measured in healthy human serum samples spiked with standard peptide, and calculated as the percentage recovery of serum in buffer.

Lower limit of measurement range (LLMR) and upper limit of measurement range (ULMR) was calculated based on the 10 individual standard curves from the intra- and inter-assay variation.

### Biological Validation of PRO-C28 as a Biomarker for Cancer

PRO-C28 was measured in serum samples from two cohorts of healthy controls (obtained from Lee Biosolutions, USA and Valley BioMedical, USA), in serum samples from a cohort of patients with various cancer types (obtained from Asterand, USA, and including samples from patients with various adenocarcinomas, infiltrating ductal carcinoma of the breast, malignant melanoma of the skin and small/squamous carcinoma of the lung) and in serum samples from a cohort of lung cancer patients (obtained from ProteoGenex, USA). PRO-C28 levels were measured blinded, using the PRO-C28 ELISA protocol described above. All serum samples were collected after informed consent and approval by the local Ethics Committee, and all serum samples from cancer patients were collected prior to resection. The patient demographics are shown in Tables 1 and 2. Although the average age of the patients in one of the healthy cohorts (the samples obtained from Lee Biosolutions) was significantly different to the average age of the patients in the two cancer cohorts, there was no correlation between age and PRO-C28 levels in any of the cohorts.

TABLE 1
Patient demographics for all cohorts

| | Lee | Valley | Asterand | Proteogenex |
|---|---|---|---|---|
| | Biosolutions | BioMedical | (cancer | (lung |
| | (Healthy) | (Healthy) | mixed) | cancer) |
| n | 54 | 43 | 85 | 40 |
| Age (SD) | 39 (11.0) | 72 | 60 (11.2) | 63.75 (3.6) |
| Gender, | 24/30 | | 42/43 | 21/19 |
| (M/F) | | | | |
| BMI mean | NA | | 26.1 (5.4) | 25.2 (4.8) |
| (SD) | | | | |
| NYHA | NA | | NA | NA |
| stage (II/III) | | | | |
| Ejection | NA | | NA | NA |
| fraction, % | | | | |
| (SD) | | | | |

TABLE 2
Patient demographics for the cancer cohorts as
combined and then broken down by cancer type

| Group | n | Age, median |
|---|---|---|
| Colorectal cancer | 7 | 63 |
| Lung cancer | 60 | 63 |
| Ovarian cancer | 10 | 53 |
| Pancreas cancer | 5 | 67 |
| Prostate cancer | 14 | 63 |
| Gastric cancer | 9 | 69 |
| Breast cancer | 13 | 56 |
| Malignant melanoma | 7 | 43 |

### Biological Validation of PRO-C28 as a Prognostic Marker in Pancreatic Cancer

PRO-C28 was measured in pre-treatment serum samples from a cohort of 701 patients with stage I-IV pancreatic cancer (PC). All PC patients were from the Danish BIOPAC (BIOmarkers in patients with Pancreatic Cancer) study (NCT03311776). Patients were recruited from six Danish hospitals from December 2008 until September 2017. PC patients had histologically confirmed tumors and were treated with various types of chemotherapy according to national guidelines (www.gicancer.dk). The study was carried out in accordance with the recommendations of the Danish Regional Committee on Health Research Ethics. The BIOPAC protocol was approved by the Danish Regional Committee on Health Research Ethics (VEK ref. KA-20060113) and the Data Protection Agency (j.nr. 2006-41-6848). All subjects gave written informed consent in accordance with the Declaration of Helsinki, version 8. Serum samples were obtained at the time of diagnosis or before operation. Samples were processed according to nationally approved standard operating procedures for blood (www.herlevhospital.dk/biopac.dk).

### Results

### Clone Selection and Characterization

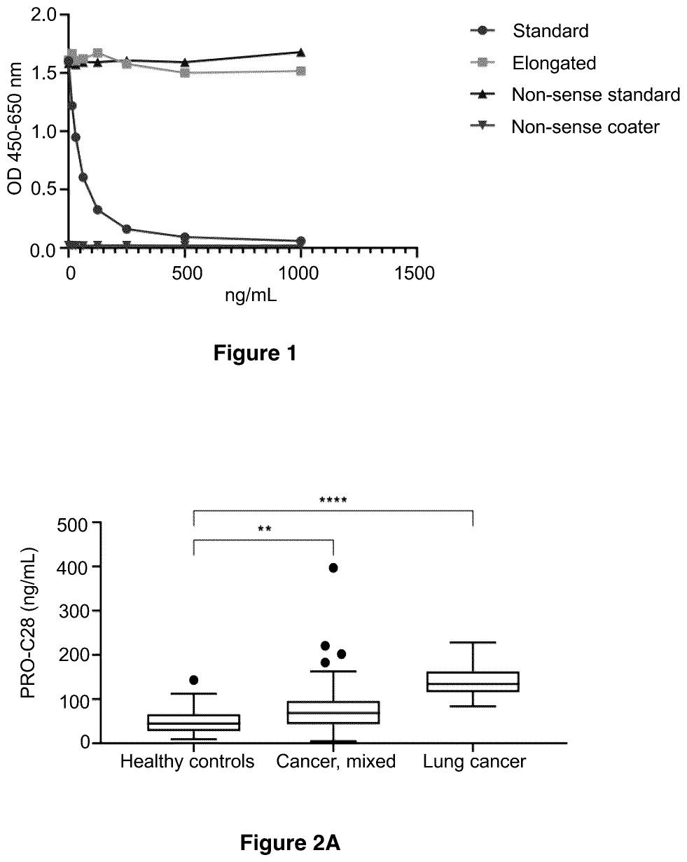

The best antibody producing hybridomas were screened for reactivity and selectivity towards the standard peptide, and based on reactivity the clone NBH218#65 8C11-2F10-1H7 was chosen and used for production of monoclonal antibodies targeting PRO-C28 for use the technical and biological evaluation of the PRO-C28 ELISA. The monoclonal antibodies were of the isotype: IgG2b, k. No reactivity was found towards the elongated peptide, non-sense peptide or non-sense coater (FIG. 1).

### Technical Evaluation of the PRO-C28 ELISA

A series of technical validations were performed to evaluate the PRO-C28 ELISA assay. A summary of the validation data is shown in Table 3.

TABLE 3
technical characteristics of the PRO-C28 competitive ELISA

| | |
|---|---|
| Intra/inter variation | Intra-variation: 6% |
| | Inter-variation: 14% |
| Dilution recovery | Serum: 112% (mean) |
| | EDTA plasma: 99% (mean) |
| | Heparin plasma: 88% (mean) |
| | Citrate plasma: 94% (mean) |
| | Urine: 99% (mean) |
| Spiking recovery | 92% (mean) |
| (peptide in serum) | |
| Measurement range | 9.5-282 ng/ml |
| Ic50 | 53.2 ng/ml |

### Biological Evaluation of PRO—C28 as a Biomarker for Cancer

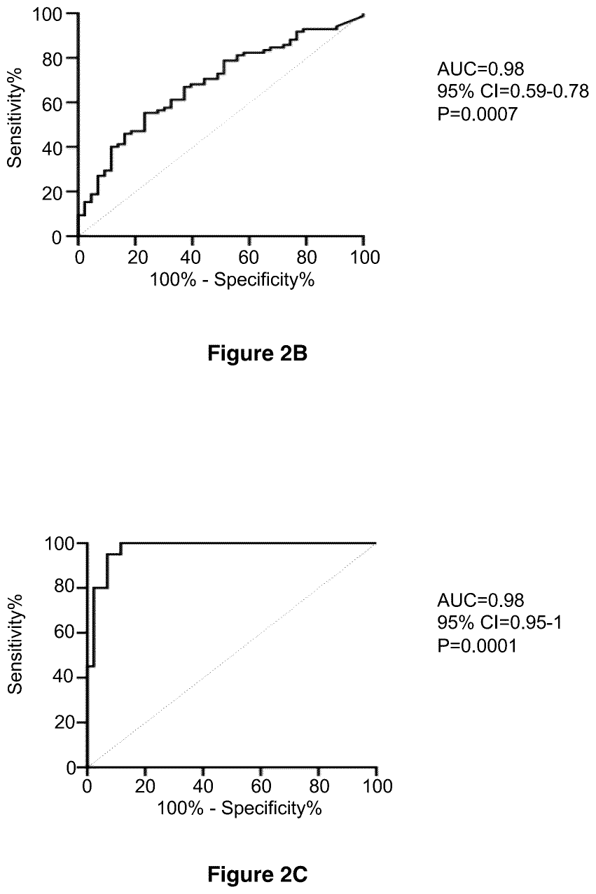

As shown in FIG. 2A, levels of PRO-C28 were significantly elevated in the cohort of patients with mixed types of cancers and in the cohort of lung cancer patients as compared to the cohort of healthy controls (Lee Biosolutions cohort). PRO-C28 levels were assessed to be on average 49.77 ng/ml (30.37) in healthy controls (Lee Biosolutions cohort), 78.32 ng/ml (55.92) in the mixed cancer patient cohort and 140.8 ng/ml (34.33) in the lung cancer patient cohort (control vs. mixed P=0.002, control vs. lung P<0.0001). PRO-C28 significantly discriminated between healthy and mixed cancer (FIG. 2B AUC=0.68, P=0.0007) and healthy and lung cancer (FIG. 2C, AUC=0.98, P<0.0001). Hence, PRO-C28 can be used to discriminate between healthy patients and cancer patients of various etiology with high accuracy and significance.

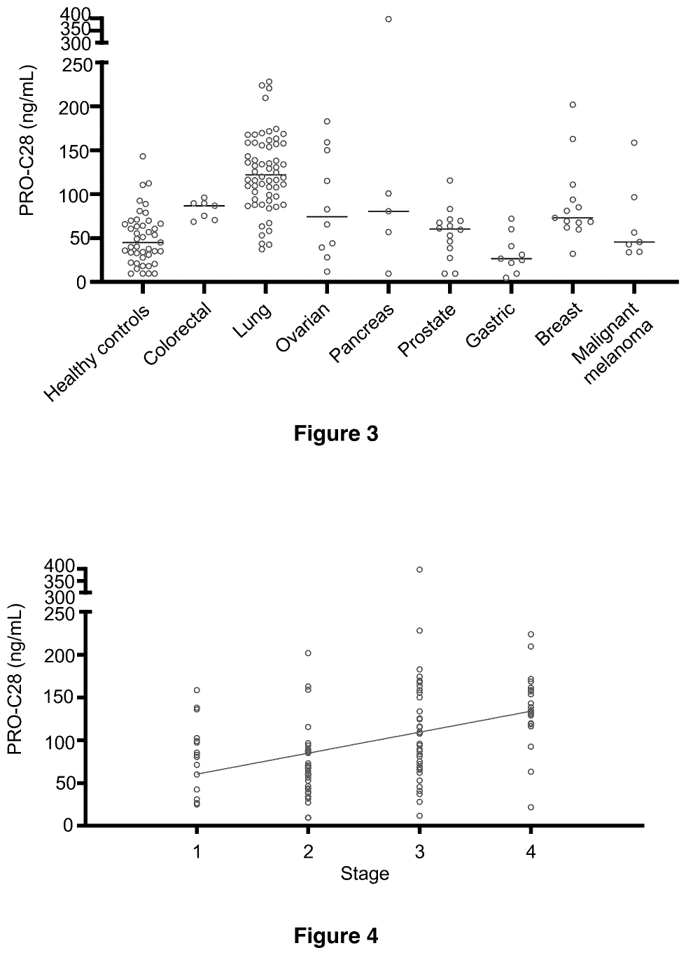

The results broken down by cancer type are shown in FIG. 3 and Table 4. Lung cancer and breast cancer patients had significantly elevated levels of PRO-C28 as compared to healthy controls (Valley BioMedical cohort), when evaluated by either of the statistical analyses used (the Dunn's multiple comparisons test or Mann Whitney test). Colorectal cancer was also significantly elevated according to the Mann Whitney test, with a trend for ovarian cancer and pancreatic cancer being elevated as well.

TABLE 4
statistical analyses

| | Dunn's multiple | Mann Whitney |
|---|---|---|
| Comparison | comparisons test | test |
| (Healthy control vs.) | (Adjusted P Value) | (p-value) |
| Colorectal | 0.2314 | 0.0011 |
| Lung | <0.0001 | <0.0001 |
| Ovarian | 0.2983 | 0.069 |
| Pancreas | 0.9328 | 0.1705 |
| Prostate | >0.9999 | 0.4003 |
| Gastric | >0.9999 | 0.1050 |
| Breast | 0.0574 | 0.0008 |
| Malignant melanoma | >0.9999 | 0.4061 |

To evaluate the association between stage of disease and PRO-C28, the cancer patient results were also grouped according to whether the patient had Stage 1 (n=15), stage 2 (n=35), stage 3 (n=40) or stage 4 (n=22) cancer, not taking into the account the 13 patients where stage-information was not available. The PRO-C28 levels of the patients in each of these groups are shown in FIG. 4 and are statistically summarized in Table 5. As shown, PRO-C28 levels correlate with stage of disease, further suggesting that PRO-C28 levels are associated with tumor burden and hence potentially also prognosis.

TABLE 5
regression analysis to describe the association between PRO-C28 and stage
Regression Equation, y = 1.9589 + 0.006574 x

| Parameter | Coefficient | Std. Error | 95% CI | t | P |
|---|---|---|---|---|---|
| Intercept | 1.9589 | 0.1651 | 1.6316 to | 11.8617 | <0.0001 |
| | | | 2.2862 | | |
| Slope | 0.006574 | 0.001430 | 0.003740 to | 4.5973 | <0.0001 |
| | | | 0.009408 | | |

| | |
|---|---|
| F-ratio | 21.1353 |
| Significance level | P < 0.0001 |

### Biological Evaluation of PRO-C28 as a Prognostic Marker in Pancreatic Cancer

Kaplan Meier curves and the log-rank p-value were used to compare the overall survival (OS) curves of the cohort of pancreatic cancer patients, with the patients being divided into tertiles (Q1, Q2, and Q3) according to pretreatment levels of PRO-C28. A p-value p<0.05 was considered statistically significant. A univariate Cox proportional-hazard regression model was to calculate the hazard ratios (HR) with 95% confidence interval (CI) for the OS per PRO-C28 biomarker tertile: Q2 and Q3 relative to Q1.

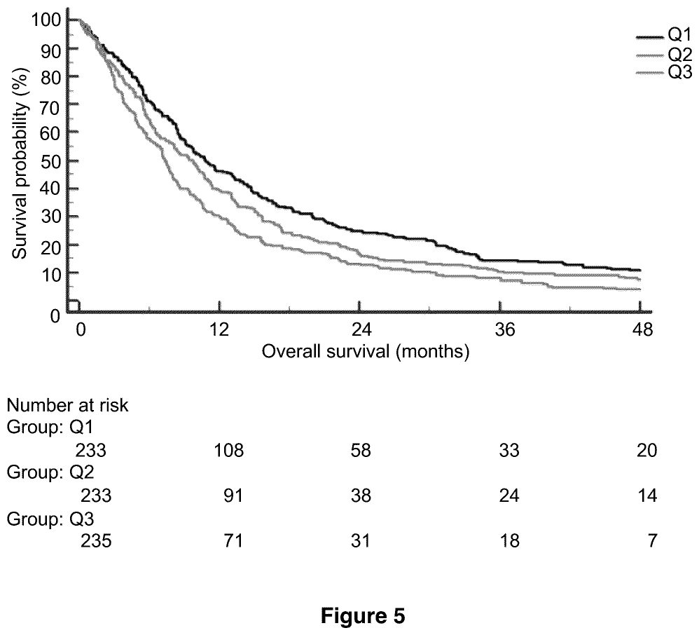

As shown in Table 5 and FIG. 5, when evaluating the association between pre-treatment levels of PRO-C28 and OS, ‘high’ PRO-C28 levels (Q3) and ‘medium’ PRO-C28 levels (Q2) were predictive of shorter OS compared to ‘low’ PRO-C28 levels (Q1), with patients in Q2 and Q3 having 53% (HR=1.53) and 24% (HR=1.24) increased risk of dying compared to patients in Q1 (HR=1.0). Likewise, the Kaplan Meier curves for each tertile were significantly different (log rank p-value <0.0001), and the median OS time decreased numerically from Q1 to Q2 to Q3. These data indicate that PRO-C28 has prognostic value in patients with PC.

TABLE 5

| PRO-C28 | Q1 | Q2 | Q3 |
|---|---|---|---|
| Events/total | 216/233 | 224/233 | 230/235 |
| Median survival (months) | 10.8 | 9.7 | 7.4 |
| HR (95% CI) | | 1.24 | 1.54 |
| | | (1.03-1.50) | (1.28-1.90) |

| | |
|---|---|
| P-value (log-rank) | <0.0001 |

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### References

1. Gebauer, J. M., Kobbe, B., Paulsson, M. & Wagener, R. Structure, evolution and expression of collagen XXVIII: Lessons from the zebrafish. Matrix Biol. 49, 106-119 (2016).
2. Veit, G. et al. Collagen XXVIII, a novel von Willebrand factor A domain-containing protein with many imperfections in the collagenous domain. J. Biol. Chem. 281, 3494-3504 (2006).
3. Annis, D. S., Mosher, D. F. & Roberts, D. D. NIH Public Access. 27, 339-351 (2009).
4. Schiller, H. B. et al. Time- and compartment-resolved proteome profiling of the extracellular niche in lung injury and repair. Mol. Syst. Biol. 11, 819-819 (2015).
5. Lai K K Y, Shang S, Lohia N, Booth G C, Masse D J, Fausto N, et al. (2011) Extracellular Matrix Dynamics in Hepatocarcinogenesis: a Comparative Proteomics Study of PDGFC Transgenic and Pten Null Mouse Models. PLoS Genet 7(6): e1002147. doi.org/10.1371/journal.pgen.1002147

1. A method of immunoassay for detecting and/or monitoring a cancer in a patient, the method comprising
(i) contacting a biofluid sample from a patient with a monoclonal antibody that specifically binds to a C-terminal epitope of type XXVIII collagen,
(ii) detecting and determining the amount of binding between said monoclonal antibody and peptides in the sample, and
(iii) correlating said amount of binding of said monoclonal antibody as determined in step (ii) with values associated with normal healthy subjects and/or values associated with known cancer severity and/or values obtained from said patient at a previous time point and/or a predetermined cut-off value.

2. The method of claim 1, wherein the cancer is lung, breast, colorectal or pancreatic cancer.

3. The method of claim 2, wherein the method is a method for detecting the stage or determining the prognosis of a cancer in a patient.

4. The method of claim 1, wherein said monoclonal antibody specifically binds to a C-terminus amino acid sequence QETCIQG (SEQ ID NO: 1).

5. The method of claim 4, wherein said monoclonal antibody does not recognize or specifically bind to an elongated version of said C-terminus amino acid sequence which is QETCIQGA (SEQ ID NO: 2).

6. The method of claim 4, wherein said monoclonal antibody does not recognize or specifically bind to a truncated version of said C-terminus amino acid sequence which is QETCIQ (SEQ ID NO: 3).

7. The method of claim 4, wherein the monoclonal antibody is raised against a synthetic peptide having the amino acid sequence QETCIQG (SEQ ID NO: 1).

8. The method of claim 1, wherein said biofluid is blood, serum, plasma, urine or a supernatant from cell or tissue cultures.

9. The method of claim 1, wherein said immunoassay is a competition assay or a sandwich assay.

10. The method of claim 1, wherein said immunoassay is a radioimmunoassay or an enzyme-linked immunosorbent assay.
